(19) United States
(12) Patent Application Publication
(10) Pub. No.: US 2005/0085924 A1
(43) Pub. Date: Apr. 21, 2005

(54) Tissue infiltratable prosthetic device incorporating an antimicrobial substance

(76) Inventors: Roger E. Darois, Foster, RI (US); Stephen N. Eldridge, Exeter, RI (US); Donna S. Torres, Attleboro, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/688,209

(22) Filed: Oct. 17, 2003

(51) Int. Cl. A61F 2/02

(52) U.S. Cl. 623/23.74

(57) ABSTRACT

The invention relates to prosthetic devices that include antimicrobial substances for reducing microbial infection, and method of using such implants for repairing, reconstructing and/or augmenting a defect or a weakness in a wall of a muscle, tissue or organ.

### FIELD OF THE INVENTION

The invention relates to a prosthetic device that is infection resistant and tissue infiltratable.

### BACKGROUND OF THE INVENTION

Various prosthetic devices have been implanted in the human body to repair, replace and/or augment a defect or weakness in a tissue or muscle wall. Representative is BARD MESH flat sheet, a knitted polypropylene monofilament fabric that encourages tissue ingrowth around and through the fabric, resulting in an integrated sheath of fabric and tissue that reinforces and repairs the damaged anatomical structure. Particularly in the repair of ventral hernias, where the prosthetic repair fabric may be exposed to sensitive neighboring organs and tissue, like the intestine, it may be desirable to isolate the porous repair fabric from the abdominal viscera to reduce the incidence of post-operative adhesions. In the BARD COMPOSIX mesh family of products, a barrier layer of submicronal expanded polytetrafluoroethylene (ePTFE), which does not promote tissue ingrowth, is united with a layer of BARD MESH sheet, forming a composite prosthetic repair fabric having a tissue ingrowth side and a barrier side. The BARD COMPOSIX mesh is implanted so that the barrier side is positioned between the porous fabric and any areas of potential postoperative adhesion.

Surgical site infections occur in about 5-7% of ventral hernia cases and about 1-2% of inguinal hernia cases. It is a common misconception, however, that prosthetic devices cause post-operative infections. An infection is caused by introduction of bacteria to the surgical site from either internal or external sources.

Depending upon the health of the patient, the severity of the infection and other factors, the infection can often times be treated with systemic antibiotics. Less frequently the mesh may have to be removed during a re-operation due to the ineffectiveness of systemic treatment, and the infection treated with antibiotics. Removal of the mesh can be difficult and it leaves the patient with no reinforcement for the defect.

Antimicrobial agents have been incorporated in ePTFE fabrics and other prosthetic repair devices to increase the likelihood of successful systemic treatment without mesh removal. For example, it is known to employ silver and/or silver salts in hernia repair devices. Although effective at reducing the incidence of bacteria colonization, it has been discovered by the present inventors that completely coating a soft tissue repair device with an antimicrobial substance may unexpectedly impair desired tissue ingrowth into and/or around the prosthetic device.

It is an object of the present invention to provide a prosthetic device that will inhibit microbial growth within the intestines and adjacent to the device.

### SUMMARY OF THE INVENTION

The present invention is a prosthetic device that includes one or more antimicrobial substances. The prosthetic device has particular application in repairing, reconstructing and/or augmenting a defect or a weakness in a wall of a muscle, tissue or organ. The invention also includes methods for using such prosthetic devices in repairing, reconstructing and/or augmenting a defect or a weakness in a wall of a muscle, tissue or organ.

In one embodiment of the invention, the prosthetic device includes a first portion that is tissue infiltratable, a second portion that is a barrier to tissue ingrowth; and an effective amount of at least one antimicrobial substance incorporated with said second portion. The effective amount of the antimicrobial substance is not incorporated with said first portion.

In another embodiment of the invention, the prosthetic device includes a flexible, biocompatible and implantable porous fabric sheet having a first surface and a flexible, biocompatible and implantable substantially non-porous barrier sheet having a second surface. The porous fabric sheet and the barrier sheet are permanently attached together with the first surface facing the second surface. The prosthetic device in this embodiment also includes an effective amount of at least one antimicrobial substance incorporated with the substantially non-porous barrier sheet. The porous fabric sheet does not incorporate an effective amount of said at least one antimicrobial substance.

In a third embodiment of the invention, the prosthetic device includes a first portion that is tissue infiltratable and a second portion that is a barrier to tissue ingrowth. The prosthetic device also incorporates an effective amount of at least one antimicrobial substance that does not detrimentally impair tissue ingrowth into and/or around the tissue infiltratable first portion.

Other objects and features of the present invention will become apparent from the following detailed description when taken in connection with the accompanying drawings. It is to be understood that the drawings are designed for the purpose of illustration only and are not intended as a definition of the limits of the invention.

### BRIEF DESCRIPTION OF THE FIGURES

FIG. 1 is a perspective view of a prosthetic device according to a first embodiment of the present invention.

### DETAILED DESCRIPTION OF THE INVENTION

For ease of understanding, and without limiting the scope of the invention, the prosthetic device to which this patent is addressed is described below particularly in connection with a composite prosthesis for use in the repair of ventral hernias. The invention is not so limited, however, and it is contemplated that the present invention may be suitable for the repair, reconstruction and/or augmentation of other anatomical weaknesses or defects, such as occur in other soft tissue and muscle walls, as well as in other body parts.

The present inventors have investigated the effects of antimicrobial substances on tissue ingrowth in composite prosthetic devices. The inventors have determined that tissue ingrowth may be detrimentally impacted when the entire composite prosthetic device is coated with an antimicrobial substance. The inventors have discovered, however, that satisfactory tissue ingrowth properties and desired antimicrobial affect can be achieved by incorporating an effective amount of an antimicrobial substance exclusively in the barrier portion of the prosthetic device. It also is contemplated that an effective amount, or less, of an antimicrobial substance may be incorporated in the tissue infiltratable layer, so long as it is no more detrimental to tissue infiltration than is the effective amount of antimicrobial substance incorporated in the barrier layer. For purposes of this specification and claims, “effective amount” means at least a sufficient quantity of antimicrobial substance to inhibit the growth of microbes within and adjacent to the device. The testing also can be carried in vitro using standard methods such as zone of inhibition assays, some of which are described in greater detail in the Examples. Further, “incorporated” or “incorporating” means an antimicrobial substance(s) that is provided on and/or within a prosthetic device, and also includes a portion or layer of a prosthetic device that is formed of a material that is inherently antimicrobial (e.g., a fabric formed of silver wire).

The effective amount of antimicrobial substances does not detrimentally impair tissue ingrowth into and/or around the prosthetic devices. As used herein, tissue ingrowth is not detrimentally impaired when the ingrowth is sufficient for the medical application that the prosthetic device is used for. As will be appreciated by one of ordinary skill in the art, greater or lesser amounts of tissue ingrowth may be required for successful medical use of the prosthetic devices of the invention. In preferred embodiments, the tissue ingrowth will not be substantially impaired relative to an untreated prosthetic.

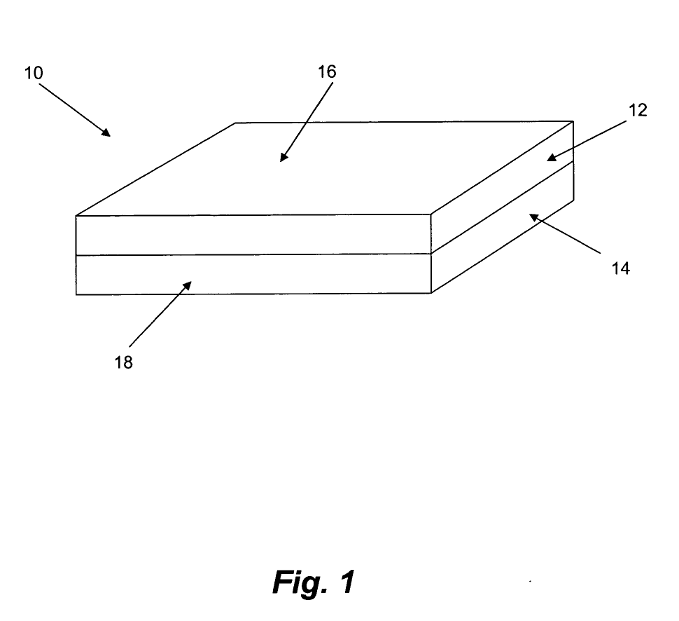

A composite prosthesis 10 according to the present invention is illustrated in FIG. 1 and includes a first tissue infiltratable layer 14, such as a knitted fabric sheet, and a second barrier layer 12 that is resistant to tissue ingrowth, such as a substantially non-porous or sub-micronal porous sheet. The two sheets may be arranged face-to-face, so that the barrier covers at least a part of the tissue infiltratable layer. As illustrated, the two layers may be approximately the same size and permanently attached together. An effective amount of an antimicrobial substance 16 is incorporated with the barrier layer to inhibit growth of microbes within or adjacent to the device. The tissue infiltratable layer does not include an antimicrobial substance. Alternatively, an amount or type of an antimicrobial substance 18 may be incorporated into the tissue infiltratable layer that is no more detrimental to tissue ingrowth as compared to the amount or type of antimicrobial substance that is incorporated into the barrier layer.

For purposes of this specification and claims, tissue ingrowth is not detrimentally impaired if a prosthetic repair device incorporating an antimicrobial substance is characterized by tissue ingrowth of at least about 10% of the amount of tissue ingrowth that would occur in the same structure absent the antimicrobial substance.

In certain embodiments, the antimicrobial substance may be released in a controlled manner and/or for a prolonged period of time. Controlled release means that the antimicrobial substance is steadily released from the prosthetic device over time. As used herein, a prolonged period of time is at least 5 days.

In some embodiments, the antimicrobial substance may be present in an amount that produces a zone of microbial growth inhibition or bacteriostasis of greater than or equal to about 0.5 mm, more preferably at least about 1 mm, still more preferably at least about 5 mm, and most preferably at least about 10 mm. The zone of microbial growth inhibition can be tested using standard methods of testing, such as by measuring in vitro the zone of microbial growth inhibition that surrounds a piece of material used in forming the antimicrobial prosthetic device of the invention. Exemplary methods for testing the zone of microbial growth inhibition, particularly the zone of bacterial growth inhibition, are provided herein.

The skilled artisan can select the preferred arrangement of the portions of the prosthetic device and the levels of the antimicrobial substance to achieve a preferred balance between antimicrobial effect and tissue ingrowth. To avoid detrimental affect on tissue infiltration, in some embodiments the antimicrobial substance will be incorporated with the prosthetic device such that there is a minimum distance between the antimicrobial substance of the prosthetic device and the nearest or farthest surface of the prosthetic that is porous or tissue infiltratable. To obtain the least effect on tissue infiltration, the minimum distance between the nearest surface of the prosthetic device and the antimicrobial substance will be equal to or greater than the zone of inhibition for the particular amount of antimicrobial substance incorporated in the prosthetic device, such that the entire porous or tissue infiltratable portion is outside of the zone of inhibition. In other embodiments, the zone of inhibition will be the minimum distance between the antimicrobial substance and the farthest surface of the porous or tissue infiltratable portion of the prosthetic device. For maximal antimicrobial effect, the porous or tissue infiltratable portion of the prosthetic device will lie entirely within the zone of inhibition.

In yet other embodiments, the antimicrobial substance in the medical implant is present in an amount that produces zones of bacterial growth inhibition for sustained periods of time, preferably at least about 5 days. More preferably still, the antimicrobial substance is present in an amount that produces zones of bacterial growth inhibition or is bacteriostatic for at least about 10 days, more preferably at least about 14 days, still more preferably at least about 18 days, and yet more preferably at least about 21 days.

The antimicrobial substances used in the implants of the invention may include antibiotics, antifungal substances and antiviral substances.

In certain embodiments, the antimicrobial substance is an antimicrobial metal or an antimicrobial metal salt. The antimicrobial metal or antimicrobial metal salt may be selected from the group consisting of: silver (Ag), gold (Au), platinum (Pt), palladium (Pd), iridium (Ir), tin (Sn), copper (Cu), antimony (Sb), bismuth (Bi), zinc (Zn), mercury (Hg), lead (Pb), cadmium (Cd), chromium (Cr), and thallium (Ti), alloys or substances containing one or more of said metals or metal salts, and mixtures of any combination of any or all of the foregoing.

In one embodiment, the antimicrobial substance is silver or a silver salt. Suitable silver salts include but are not limited to: silver chloride, silver nitrate, silver iodide, silver citrate, silver lactate, silver acetate, silver propionate, silver salicylate, silver bromide, silver ascorbate, silver laurel sulfate, silver phosphate, silver sulfate, silver oxide, silver benzoate, silver carbonate, silver sulfadiazine, and silver gluconate.

The antimicrobial silver incorporated with the prosthetic device may be characterized by the level of ammonia soluble (“available”) silver, and the level of total silver. It is the available silver that dissolves most readily, and it is believed that the available silver provides the antimicrobial function. The remaining part of the total silver typically contributes to the overall structure of the silver applied to the prosthetic, e.g., a silver coating. A silver application with a very high percentage of available silver (i.e. >70%) has greater antimicrobial efficacy, but perhaps lesser mechanical properties, than a coating with a lower percentage of available silver. Methods for analyzing total and available silver content of a barrier layer are known to one of ordinary skill in the art; exemplary methods are provided in the Examples below.

Other suitable antimicrobial substances include, but are not limited to: biguanide substances, such as chlorhexidine and its salts, triclosan, penicillins, tetracyclines, aminoglycosides, such as gentamicin and tobramycin, polymyxins, rifampicins, bacitracins, erythromycins, vancomycins, neomycins, chloramphenicols, miconazole, quinolones, such as oxolinic acid, norfloxacin, nalidixic acid, pefloxacin, enoxacin, and ciprofloxacin, sulfonamides, nonoxynol 9, fusidic acid, cephalosporins, and combinations of such substances and similar substances.

Antibiotic ceramic particles useful with the present invention include zeolites, hydroxyapatite, zirconium phosphates or other ion-exchange ceramics. Hydroxyapatite particles containing antimicrobial metals are described, e.g., in U.S. Pat. No. 5,009,898. Zirconium phosphates containing antimicrobial metals are described, e.g., in U.S. Pat. Nos. 5,296,238; 5,441,717; and 5,405,644. Antibiotic zeolites may be prepared by replacing all or part of the ion-exchangeable ions in zeolite with ammonium ions and antibiotic metal ions, as described in U.S. Pat. Nos. 4,938,958 and 4,911,898. Such zeolites may be incorporated in antibiotic resins (see U.S. Pat. Nos. 4,938,955 and 4,906,464) and polymer articles (see U.S. Pat. No. 4,775,585). Polymers including the antibiotic zeolites have been used to make different kinds of articles, see U.S. Pat. Nos. 5,714,445; 5,697,203; 5,562,872; 5,180,585; 5,714,430; and 5,102,401.

Polymers may be employed in application of the antimicrobial substances to the prosthetic device in accordance with the present invention, including hydrophilic polymers, hydrophobic polymers, and mixtures of these two types of polymers. The use of hydrophilic polymers may be desirable to increase lubricity for patient comfort, to increase absorption of aqueous fluids from the body which aids in the release of the antimicrobial substances (e.g., metal ions), to inhibit bacterial attachment, and to improve solubility for some metal salts. Hydrophilic polymers particularly suited to the invention are those that are soluble in water or in organic solvents containing water. The ability to add water to the polymer composition without precipitating the polymer allows the addition of water-soluble salts directly to the coating composition. Water facilitates the formation of salt colloids within the polymer composition. For this reason, it is preferred that the polymer solution contain from 1 to 50% water by weight, more preferably from 5 to 30% water.

However, the use of water is not limiting, as salt colloids can also be formed using alcohols, organic solvents, or both that contain little or no water. The use of alcohols and organic solvents containing from 0 to 1% water are preferred when hydrophobic polymers are employed in the present invention.

Examples of hydrophilic polymers which may be used in antimicrobial applications include, but are not limited to, polyurethanes, including polyether polyurethanes, polyester polyurethanes, polyurethane ureas, and their copolymers; polyvinylpyrrolidones; polyvinyl alcohols; polyethylene glycols and their copolymers; polypropylene glycols and their copolymers; polyoxyethylenes and their copolymers; polyacrylic acid; polyacrylamide; carboxymethyl cellulose; cellulose and its derivatives; dextrans and other polysaccharides; starches; guar; xanthan and other gums and thickeners; collagen; gelatins; and other biological polymers. Preferred hydrophilic polymers are polyurethanes and polyurethane copolymers, such as polyether polyurethaneurea.

Examples of hydrophobic polymers suitable for use in the antimicrobial substance applications of the present invention include, but are not limited to, polytetrafluoroethylene, polyvinyl chloride (PVC), polyvinylacetate, poly(ethylene terephthalate), silicone, polyesters, polyamides, polyureas, styrene-block copolymers, polymethyl methacrylate, acrylic-butadiene-styrene copolymers, polyethylene, polystyrene, polypropylene, natural and synthetic rubbers, acrylonitrile rubber, and mixtures and copolymers of any of the above. The preferred hydrophobic polymer depends upon the substrate to be coated. Hydrophobic polymers that are chemically similar or identical to the substrate are advantageously used alone or in combination with hydrophilic polymers to form coatings that enhance adhesion of the coating to the substrate.

In some instances, the polymers used in antimicrobial applications are biodegradable polymers. Suitable biodegradable polymers include the homopolymers poly(glycolic acid), poly(D-lactic acid), poly(D,L-lactic acid), poly(D,L-ethylglycolic acid), poly(dimethylglycolic acid), poly(D,L-methylethylglycolic acid), and poly(E-caprolactone), polylactic acid (PLA), as well as biodegradable polyhydroxy butyric acid and mixtures thereof.

Inclusion of a biodegradable polymer such as PLA in the matrix can yield prolonged antimicrobial activity. The initial burst of drug which occurs during the first few hours or days after implantation is reduced or eliminated since the drug is bound in the biodegradable polymer and will be released only when degradation of the polymer occurs.

The same principles, methods, and choice of application of antimicrobial substances as described above also apply to therapeutic substances that can be used in the prostheses of the invention. Therapeutic substances that can be used include antithrombogenic substances, anesthetics, anti-inflammatory substances, analgesics, anticancer substances, vasodilation substances, wound healing substances, angiogenic substances, angiostatic substances, immune boosting substances, growth factors, and other biological therapeutic substances. The present invention can also encompass any combination of the aforementioned or additional therapeutic substances that are known to one of ordinary skill in the art.

An antimicrobial substance may be introduced during the production of a starting material that will be used to form a barrier portion, whether it be a filament that is knit, woven, braided, or otherwise interlaced into a sheet or other construct, or a solution, suspension or slurry that is expanded, cast, extruded, or otherwise fabricated into a sheet or other construct. An antimicrobial substance may be applied after construction of the barrier portion or sheet but prior to juxtaposition or attachment to another portion or layer of the prosthetic device. In other embodiments, the various layers or portions of the prosthetic device may be assembled together and then an antimicrobial substance may be applied to one or more of such portions or layers. The point in time when the antimicrobial substance is incorporated with the prosthetic device is not necessarily critical to the present invention.

Contemplated methods for incorporating an antimicrobial substance include, but are not limited to, coating (including spraying, dipping, rolling, sputtering, depositing), impregnating, implanting, etching, and other methods as should be apparent to one of skill in the art. In one representative technique, described below, an antimicrobial metal or metal salt coating is applied by vacuum deposition to the barrier layer. The processing conditions may be controlled to produce a coating characterized by a nanocrystalline structure that allows for sustained release of antimicrobial metal ions when contacted with body fluids. In another representative approach, a portion of a prosthetic device is soaked in a solution, slurry or suspension of an antimicrobial substance(s). The solution or suspension may be prepared at room temperature or at a slightly elevated temperature with, if desired, the aid of agitation, stirring, or mixing. Solvents may be selected that evaporate from the coating at room temperature, or that evaporate at an elevated temperature. Reduced atmospheric pressure and other methods that aid in drying may be used. An application of an antimicrobial substance may be repeated one or more times until a desired thickness is reached. Further, different antimicrobial substances may be administered simultaneously, or during different coating treatments. The characteristics of the antimicrobial coating may be varied depending upon the desired application as should be apparent to one of skill in the art. Thus, it is contemplated that the following features of an antimicrobial substance, amongst others, may be adjusted: thickness, composition, effectiveness, and physical properties.

The invention does not require complete incorporation of an antimicrobial substance to the prosthetic device. For example, where an antimicrobial substance is applied to a layer having a sheet-like configuration with a top surface and a bottom surface, the antimicrobial substance may be provided only on the top or the bottom surface. Or a particular layer may be dipped into an antimicrobial substance so that both the top and bottom surface become coated, as may some but not all of the thickness of the particular layer. Similarly, the antimicrobial substance may be applied to only a segment of a layer, viewed in a direction from one edge towards the other. The antimicrobial substance also may be dispersed in various spaced locations about the layer, whether uniformly distributed or otherwise.

In certain embodiments of the invention, the antimicrobial substance(s) are applied in powder form directly to one or more layers of the prosthetic device. The antimicrobial substance may be applied directly to the surface of the layer or may be applied after application of, or simultaneously with, an adhesive or bonding agent. Alternatively, the layer may be treated chemically or mechanically to promote adherence of the powder to the prosthetic device.

The prosthetic device may have any shape or size that is indicated for the particular procedure for which it will be employed. Although as illustrated the device has a generally rectangular shape, other shapes are contemplated including, but not limited to, circles, ovals, squares, and irregular configurations. The implant may be in the form of an essentially sheet-like patch having a flat, convex, concave, convex/concave, or other shape, and may take the form of a more complex three-dimensional shape, such as a cylinder or cone. The implant may be pliable, facilitating placement and/or conformance to the surgical site. The prosthetic device may be sized to cover part or, preferably, all of the defect.

Although illustrated and described above as including two separate layers, the inventive prosthesis may be formed of a single layer, of the same material or a combination of different materials, with a first portion that is resistant to tissue or muscle ingrowth and a second portion that is amenable to tissue infiltration. The location of tissue ingrowth sections and barrier sections need not be on opposite sides of the device, as has been shown, but may vary along an edge of the implant, a surface of the implant, and/or sections of a body portion of the implant. Either or both of the tissue infiltratable layer and the barrier layer may be formed of two or more sheets. Further, in certain embodiments the prosthetic device may include two or more tissue infiltratable layers, but not include a barrier layer, where one or more of such tissue infiltratable layers does not incorporate an antimicrobial substance while other of such layers are provided with an antimicrobial substance.

The prosthetic device may be surgically placed as an overlay, an underlay, or as a plug, and in other relationships relative to the weakened or ruptured tissue, muscle or organ as should be apparent to one of skill in the art. The prosthetic device may be flexible allowing the implant to be reduced into a narrower configuration, such as by rolling or folding, and may be inherently resilient or include resilient enabling structure so that a collapsed configuration may unfurl upon deployment. Although the invention is described in connection with a prosthetic device particularly suitable for repair of ventral hernias, the application of the inventive prosthesis is not so limited.

We now turn to a description of a prosthetic device according to the present invention that is particularly indicated for ventral hernia repair. The tissue ingrowth layer may be formed of a sheet of biologically compatible, flexible, prosthetic repair fabric having a plurality of interstices or openings which allow tissue ingrowth, integrating the repair device to host tissue after implantation. The tissue infiltratable fabric may be characterized by the strength, elongation resistance, suture retention, and handleability properties, necessary for an implantable abdominal wall repair device. A representative material is knitted polypropylene monofilament mesh, such as BARD MESH sheet (formerly known as MARLEX MESH), available from C.R. Bard, Inc. When implanted, the polypropylene mesh is believed to promote rapid tissue ingrowth into and around the mesh structure. Alternatively, other surgical materials which are suitable for tissue reinforcement and defect closure may be utilized including, without limitation, polytetrafluoroethylene (PTFE) mesh, PROLENE, SOFT TISSUE PATCH (microporous ePTFE), SURGIPRO, TRELEX, ATRIUM, MERSELENE, non-absorbable collagen, and polyester. Absorbable materials, including polyglactin (VICRYL), polyglycolic acid (DEXON), and absorbable collagen may also be employed. It also is contemplated that the fabric may be formed from monofilament or multifilament yarns which may be woven, knitted, molded, or otherwise interengaged to form the tissue infiltratable component of the implant.

A barrier portion or layer may be formed from a sheet of expanded polytetrafluoroethylene (ePTFE), having fibril lengths—also referred to as pore size or internodal distance—that will discourage tissue ingrowth. The fibril length may average less than 5 microns, less than 3 microns, less than 1 micron, or less than 0.5 microns. A representative and non-limiting sampling of other suitable barrier materials includes silicone elastomer, such as SILASTIC Rx Medical Grade Sheeting (Platinum Cured) distributed by Dow Corning Corporation, TEFLON mesh, microporous polyproplyene sheeting (CELGARD), collagen, hyaluronic acid, carboxymethyl cellulose, and glycolic acid polymers. Autogenous, heterogeneous, and xenogeneic tissue also are contemplated including, for example, pericardium and small intestine submucosa. Absorbable materials, such as SEPRAFILM and oxidized, regenerated cellulose (INTERCEED (TC7)) may be employed as well. A barrier also may be formed by treating or altering a portion of a tissue infiltratable layer to form a surface that does not promote tissue ingrowth. For example, one or more portions of a tissue infiltratable layer may be melted and resolidifed to render those portions of the layer substantially non-porous. Suitable melting techniques may include ultrasonic, induction, vibration, and infrared/laser welding, and the like. Selected regions of a tissue infiltratable layer, alternatively, may be sealed with compatible materials to prohibit tissue ingrowth. Any suitable method may be used to render selected portions of the prosthesis resistant to tissue infiltration, and other biocompatible materials may be employed, as should be apparent to one of skill in the art.

An antimicrobial substance is incorporated with the ePTFE layer. In a representative procedure, a film of submicronal ePTFE is supported in a frame and loaded into a vacuum chamber opposite a magnetron equipped with 99.99% pure silver target. The vacuum chamber is evacuated using vacuum pumps and an operating pressure of from 1-100 mTorr is established using an argon/oxygen gas mixture. The magnetron target is energized as a cathode with a potential of from 300-500 volts. Sputtering of silver is conducted until a coating of nanocrystalline silver of sufficient thickness is obtained on the barrier material. The magnetron is de-energized, and the argon/oxygen gas mixture and vacuum pumps are shut off. The vacuum chamber is vented to atmospheric pressure. The silver coated barrier layer is then removed from the vacuum chamber and separated from the supporting frame. The knitted polypropylene fabric is not subject to this silver coating treatment.

Alternatively, the ePTFE layer may be soaked in a silver or silver salt containing solution and then allowed to air dry until the solvent has evaporated, leaving a silver or silver salt coating on and perhaps in the submicronal porous network of the ePTFE sheet. The knitted polypropylene monofilament fabric is not soaked in the silver or silver salt containing solution.

The porous tissue ingrowth sheet and the antimicrobial containing submicronal ePTFE barrier sheet may remain independent and separate from each other, allowing placement one at a time of each individual sheet, if desired, at the surgical site. Once positioned relative to the tissue, muscle, or organ defect, the tissue ingrowth and barrier layers may, if desired, be joined together, using sutures (e.g., ePTFE thread), staples, biocompatible adhesive, and other fastening arrangements as should be apparent to one of skill in the art. Alternatively, the first and second layers may not be attached to each other, although each layer may separately and independently be attached by sutures, staples, biocompatible adhesive or the like, to surrounding tissue. In certain embodiments, the first and second layers may be joined together prior to implantation, either by the surgical team or by the manufacturer of the prosthetic device. The first and second layers may be joined together by a variety of techniques, as should be apparent to one of skill in the art, including without limitation, stitching, fusing, adhesive bonding, ultrasonic welding, insert molding, and stapling. The tissue infiltratable layer and the barrier layer combine together, regardless of whether they have been permanently attached to each other, to provide the desired issue ingrowth, adhesion resistance and antimicrobial effect. The prosthetic device, whether an integrally formed patch or plug including a tissue infiltratable layer and a barrier layer loaded with an antimicrobial substance, or separate sheets of these materials, is sterilized such as by exposure to a gamma ray or electron beam source and stored in a package alone or with instructions on how to use the prosthetic device in a surgical procedure.

In certain embodiments, the peripheral edge of the implant and/or the peripheral margin of the tissue infiltratable layer, may constitute a barrier so as to reduce the occurrence of adhesion formation at those locations. In a representative embodiment, the peripheral edge and margin of the tissue infiltratable layer is melted to seal the material and form an outer peripheral barrier. The barrier layer may be configured, such as with submicronal sized pores, so that a portion of the melted material of the tissue infiltratable layer becomes fused to the barrier layer. The ingrowth layer may also be stitched to the barrier layer.

Where desired to provide an erosion resistant edge, the margin and/or peripheral edge of the tissue infiltratable layer may be rendered non-porous and/or provided with a flexibility or deformability. For example, the border of the knitted fabric may be heated until the polypropylene melts and resolidifies as a solid seam. The thickness of the barrier at the margin of the tissue infiltratable layer may taper in decreasing fashion towards the peripheral edge to enhance the flexibility of the erosion resistant edge. Alternatively, a barrier material, such as ePTFE, may be wrapped over the edge of the tissue ingrowth layer and secured along the margin of the knitted surface, covering the openings at the peripheral edge and margin of the fabric. A gap left between the ePTFE wrap and the edge of the tissue infiltratable and barrier layers, may act as a deformable cushion to help resist forces of erosion.

We now describe a representative method of using a prosthetic device according to the present invention, in this case in the repair of a ventral hernia. The defect site is accessed, classically, laparoscopically, through other non-invasive technique such as a Kugel approach, or otherwise. A flexible, biocompatible substantially flat and sheet-like implant including a first tissue infiltratable layer of knitted monofilament polypropylene mesh and a second barrier layer of expanded ePTFE incorporated with an effective amount of an antimicrobial silver substance, the tissue infiltratable and barrier layers being substantially the same size and shape and joined together by ePTFE stitches, is placed at the defect opening with the barrier layer directly facing the abdominal viscera and the tissue infiltratable layer directly facing the abdominal side, so that the mesh surface is isolated from the intestines and other sensitive anatomy by the ePTFE barrier layer. The first layer of polypropylene mesh allows for rapid tissue ingrowth, and a strong repair; the second layer comprising ePTFE protects the abdominal organs and other sensitive tissues from adhesions. The antimicrobial coating on the second layer reduces the occurrence of and/or battles infections that may appear at the surgical site. If desired, the implant may be sutured, stapled or otherwise attached to surrounding anatomy.

It should be understood that the foregoing description of the invention is intended merely to be illustrative thereof and that other equivalents, embodiments and modifications of the invention may be apparent to those skilled in the art.

### EXAMPLES

We investigated a silver antimicrobial coating for an ePTFE-containing prosthesis (Bard Dulex®) for ventral hernia repair. This prosthesis, while made entirely of ePTFE, has two different sides. The minimally adhesive visceral side is smooth ePTFE with sub-micron pores. The abdominal wall side is macroporous to promote tissue ingrowth into its surface.

Materials and Methods:

Antimicrobial Silver Coating:

A coating of antimicrobial silver was applied by a vacuum deposition process that creates a nanocrystalline structure in the silver coating. This structure provides an increased surface area for silver ion release, and as a result, increased antimicrobial activity over a smooth silver surface.

The coating process allows for a wide range of antimicrobial activity. The proportion of the coating that is “available” is determined by the process conditions (sputtering current and atmosphere), while the total thickness of the coating is dependent on the sputtering current and on the time of application.

Determination of Silver Levels:

Only a percentage of the silver coating is readily soluble; the exact solubility is a complex issue and depends on the composition of the solution and particularly the pH. Laboratory tests were performed to establish the levels of ammonia-soluble silver (i.e., available silver) and total silver in a piece of implant material coated with silver under a standard set of conditions.

Square-inch pieces of the coated material were digested in an ammonia solution for several minutes. A dilution of the leachate was then analyzed by atomic absorption spectroscopy (AAS) to determine the amount of ammonia-soluble (“available”) silver present. The total silver was determined by digesting the same samples in nitric acid, which takes all of the silver into solution. A dilution of this acid solution was then also analyzed by AAS. The silver concentrations in the samples were determined by comparison with standard silver solutions. The total silver is calculated by summing the ammonia soluble silver and the acid soluble silver (J. B. Wright, et al., The Comparative Efficacy of Two Antimicrobial Barrier Dressings: In-Vitro Examination of Two Controlled Release Silver Dressings, Wounds, v. 10, n. 6, pp. 179-188).

Zone of Inhibition Testing (Plate-to-Plate Method):

The corrected zone of inhibition (CZOI) plate-to-plate test was initially used to determine the duration of antimicrobial activity of the coatings. In this test, the measured zones are “corrected” to take into account differences in the size and/or shape of the samples being tested. Bacterial lawns of Pseudomonas aeruginosa and Staphylococcus aureus were seeded on Mueller Hinton agar plates. Pieces of the antimicrobial-coated material, a control antimicrobial material (Gore DualMesh® Plus), and uncoated control material were placed on the lawns of bacteria and incubated overnight. The CZOI was calculated by measuring the total zone of inhibition across the width of the test article and then by subtracting the width of the test article. The corrected zone size was recorded, and the coated sample was then transferred to a new lawn of bacteria, and incubated. After another 24 hours, the CZOI was measured again. The sample was re-challenged with bacteria daily until no zone of inhibition was produced. The number of days that the sample continued to produce a zone of inhibition was recorded as the sample's duration of activity (J. B. Wright, et al., The Comparative Efficacy of Two Antimicrobial Barrier Dressings: In-Vitro Examination of Two Controlled Release Silver Dressings, Wounds, v. 10, n. 6, pp. 179-188).

Zone of Inhibition Testing (MHA Method):

The alternate test method used to determine the duration of activity also used Mueller Hinton agar plates and measured the corrected zone of inhibition. However, rather than remove the coated sample from the agar plate and expose it to a new lawn of bacteria each day, the sample was incubated on a non-inoculated plate of Mueller Hinton agar for a specified time period. At the end of that time period, the sample was placed onto a lawn of bacteria. After 24 hours, the CZOI was measured as described above. In this case, the duration of activity (if the sample was still active) was the number of days the sample had been incubated before exposure to the bacteria.

Implant Method (Rabbit):

Rabbits were used to test the performance of the coated implants in vivo. The rabbits were anesthetized using halothane inhalation. All procedures were conducted according to standard surgical procedures for minimizing the risk of infection in the test animals, including preparing the ventral abdominal area for surgery by clipping the hair, scrubbing the abdominal surface with chlorhexidine acetate (HIBITANE®) or another similar antimicrobial wash, followed by an alcohol rinse and use of sterile drapes covering the animal but exposing the incision area.

A midline abdominal incision was made to enable access to the lateral abdominal wall. The peritoneal surface of the lateral abdominal wall was then lightly abraded with a surgical sponge. Coated implants (25 mm×30 mm) were placed on the left and right peritoneal walls, on either side of and adjacent to the incision site, so that each animal received two implants. A space of at least 15 mm separated the implant material from the incision site. The implant material was placed with the ingrowth surface facing the peritoneal wall and secured with non-absorbable, simple, interrupted sutures at each of the four corners as well as at the mid-point of each edge. The suture knots were kept in the subcutaneous region to minimize tissue adhesions to the knots. The incisions were closed using sutures; the dermal layer were closed using subcuticular stitching to prevent the animals from chewing on their stitches. Penicillin was applied to the suture area to minimize possible incision site contamination. Tissue glue was also used to ensure that the incisions sites were well closed. The animals received an injection of butorphanal for pain management post-operatively.

All animals were weighed prior to surgery, at one week following implantation, or immediately prior to necropsy. Animals were observed daily, and any animals judged to be moribund were to be euthanized. The animals were euthanized according to days of implantation (usually 7 or 14 days). Animals were examined for any grossly observable lesions, and sharp dissection was then used to open the abdominal cavity in such a manner as to preserve the anatomical integrity of the midline structures. The presence of adhesions from the patch, patch sutures, or midline sutures was noted. If fibrous adhesions were noted, the numbers of distinct pedicles extending from the area of the patch (including patch sutures) or midline sutures were recorded. If visceral adhesions were noted, the number of distinct adhesions extending to the patch or midline sutures were counted and recorded. Photographs of the patches in situ were taken. The mesh samples with a 1 cm margin of abdominal wall were excised and placed in pre-labeled jars containing 10% neutral buffered formalin for histologic evaluation.

Implant Method (Rat):

Later implant testing of the coated composites was done using rats. The rats were anesthetized and standard surgical procedures were used, including preparing the ventral abdominal area for surgery by clipping the hair, and scrubbing the abdominal surface with povidone-iodine.

A midline abdominal incision was made to enable access to the lateral abdominal wall. A 16 mm×16 mm implant was placed to one side of the incision, with the mesh surface facing the peritoneal wall and secured with non-absorbable, simple, interrupted sutures at each of the four corners as well as at the mid-point of each edge. The suture knots were kept in the subcutaneous region to minimize tissue adhesions to the knots. The incisions were closed using sutures; the dermal layer was closed using a skin stapler.

All animals were weighed prior to surgery, at one week following implantation, or immediately prior to necropsy. Animals were observed daily, and any animals judged to be moribund were to be euthanized. The animals were euthanized according to days of implantation and examined for any grossly observable lesions. Sharp dissection was then used to open the abdominal cavity in such a manner as to preserve the anatomical integrity of the midline structures. The presence of adhesions from the patch, patch sutures, or midline sutures was noted. If fibrous adhesions were noted, the numbers of distinct pedicles extending from the area of the patch (including patch sutures) or midline sutures were recorded. If visceral adhesions were noted, the number of distinct adhesions extending to the patch or midline sutures were counted and recorded. Photographs of the patches in situ were taken. The mesh samples were excised and placed in pre-labeled jars containing 10% neutral buffered formalin for histological evaluation.

Histological Analysis:

The histological analysis was comprised of two different methodologies. The first assessment determined the nature of the inflammatory response (acute vs. chronic). The second method evaluated “incorporation” by assessment of new collagen formation.

To assess the inflammatory response, the numbers of giant cells per high power field [HPF-200 x] were counted in a pre-assigned number of fields for each slide. Giant cells were identified by their multinucleate appearance with an abundance of cytoplasm. Hematoxylin and eosin (H&E) sections were utilized for this evaluation. The number of polymorphonuclear neutrophils (PMNs) was also qualitatively assessed in the same fields, and categorized in the following groups:
1. None observed.
2. Rare (1 to 3 per HPF)
3. Occasional (4 to 10 per HPF)
4. Frequent (11 to 25 per HPF)
5. Numerous (More than 25 per HPF)

New collagen formation was evaluated using picrosirious red stained sections. The percentage of the area between a line connecting tops of each adjacent node and the underlying mesh that demonstrates polarization was estimated.

Results:

Dulex

Much of the initial testing was directed at determining the appropriate silver concentration for the coating on Bard DULEX® Mesh, and ensuring that the silver coating does not adversely affect the in vivo performance of the mesh.

Initial in vitro zone of inhibition (plate-to-plate method) testing against Pseudomonas aeruginosa and Staphylococcus aureus was used to determine a range of silver coating concentrations that would be effective for durations equal to or longer than Gore DUALMESH® Plus (Table 1). Based on that testing, a silver level of 0.211 mg/cm² available silver/0.459 mg/cm² total silver was chosen for further study. This level provided 15 days of activity versus Staphylococcus aureus compared to 6 days for DUALMESH Plus, and 4 days of activity versus Pseudomonas aeruginosa, which equaled the activity of DUALMESH Plus. DULEX mesh with that coating level was implanted in rabbits in a small pilot study. Gross observation of the implants after 14 days showed poor tissue ingrowth into the coated mesh.

TABLE 1
Zone Test Results for silver coated DULEX mesh.

| | | Total | | | |
|---|---|---|---|---|---|
| | Available | Silver | Available | | |
| | Silver | (mg/ | Silver | CZOI longevity (days) | |
| Material | (mg/cm2) | cm2) | (%) | S. aureus | P. aeruginosa |
| Dulex | 0.063 | 0.156 | 40.4% | 8 | 2 |
| Dulex | 0.109 | 0.147 | 74.1% | 12 | 3 |
| Dulex | 0.211 | 0.459 | 46.0% | 15 | 4 |
| Dulex | 0.351 | 0.433 | 81.1% | 15 | 7 |
| Dulex | 0.434 | 0.841 | 51.6% | 15 | 9 |
| Dulex | 0.791 | 0.95 | 83.3% | 15 | 14 |
| DualMesh | N/A | N/A | N/A | 6 | 4 |
| Plus | | | | | |

Because of the apparent negative effect of the originally chosen level of silver on ingrowth, we investigated a silver level for coating DULEX that would not negatively impact tissue ingrowth, including the possibility of coating one or more of the components of the composite products. Several mesh/coating variations were tried, including coatings on the barrier side of the mesh only, and coatings with a lower silver concentration on the ingrowth side of the mesh. The results of the histological analyses of DULEX mesh coated with silver on the ingrowth side are summarized in Table 2.

TABLE 2
Results of Histological Analyses of Silver-Coated
DULEX Mesh, Uncoated Control DULEX Mesh, and
DUALMESH Plus Implanted at Various Time Periods

| | Available | | | Implant | | |
|---|---|---|---|---|---|---|
| | Silver | Total Silver | Available | Duration | Inflammatory | |
| Material | (mg/cm²) | (mg/cm²) | Silver (%) | (days) | Response | Collagen Formation |
| Dulex | 0.211 | 0.459 | 46.0% | 14 | No histology, only | No ingrowth of |
| (level from | | | | | gross observation | collagen apparent |
| Table 1) | | | | | | upon observation |
| Dulex | 0.0008 | 0.014 | 5.7% | 14 | Thicker cellular response | Very little newly |
| | | | | | than control surface; | formed collagen |
| | | | | | occasional giant cells | compared to control |
| Dulex | 0.0025 | 0.014 | 17.9% | 3 | Virtually no response at | None |
| | | | | | material surface. Fibrin | |
| | | | | | network, focal areas of PMN's | |
| Dulex | 0.0025 | 0.0050 | 50.0% | 3 | Sparse macrophages. Fibrin | None |
| | | | | | network. Focal areas of PMN's | |
| | | | | | and basophilic debris | |
| Dulex | 0.0076 | 0.014 | 54.3% | 3 | Sparse to no cellular response. | None |
| | | | | | Large volumes of basophilic | |
| | | | | | debris. Focal areas of | |
| | | | | | degenerating PMN's | |
| Dulex | 0.0076 | 0.014 | 54.3% | 7 | Granulation tissue at peripheral | 30-50% of normal |
| | | | | | edge; muscle sheath heavily | |
| | | | | | infiltrated with macrophages | |
| Dulex | 0.0227 | 0.042 | 54.0% | 3 | Virtually acellular on surface; | None |
| | | | | | fibrin network; one focal area of | |
| | | | | | PMN accumulation | |
| Dulex | 0.0227 | 0.042 | 54.0% | 7 | Sparse to well populated with | 50-100% of normal |
| | | | | | macrophages; some | |
| | | | | | macrophages with abnormal | |
| | | | | | morphology | |
| Dulex | 0.068 | 0.126 | 54.0% | 7 | Material surface sparsely | Minimal |
| | | | | | populated. Pyknotic nuclei | |
| | | | | | within overlying tissue | |
| Dulex | N/A | N/A | N/A | 3 | Mixed acute & chronic; PMN & | None |
| Control | | | | | macrophages | |
| Dulex | N/A | N/A | N/A | 7 | Predominant macrophages; | Thin collagen |
| Control | | | | | infrequent giant cells | elements between |
| | | | | | | tissue and mesh |
| Dulex | N/A | N/A | N/A | 14 | Macrophages with occasional | Overlying newly |
| Control | | | | | giant cells; giant cells at “sharp” | formed thick fiber |
| | | | | | projections of ePTFE | network; newly |
| | | | | | | formed fibers in |
| | | | | | | the internodal space |
| DualMesh | N/A | N/A | N/A | 7 | Few cells, only occasional | None |
| Plus | | | | | spindle-shaped cells | |
| DualMesh | N/A | N/A | N/A | 14 | Macrophages with frequent | Fibrous tissue |
| Plus | | | | | giant cells and spindle shaped | formation parallel to |
| | | | | | cells | material surface |
| | | | | | | with some fibers |
| | | | | | | to the material |
| | | | | | | surface |

Based on previous in vitro zone testing, the lowest level implanted (0.008 mg/cm² available/0.014 mg/cm² total) could be expected to have a duration of activity of about one day against Staphylococcus aureus and Pseudomonas aeruginosa using the plate-to-plate zone of inhibition assay. However, even this lowest level of silver applied using vacuum deposition to the ingrowth side of the mesh had some impact on the inflammatory response and on the formation of collagen. The histological analysis revealed a reduction in collagen formation within the ingrowth surface of the coated mesh when compared with an uncoated control. As the silver levels increased, the effect was even more pronounced.

Composix:

As a result of the foregoing testing using DULEX mesh products, coating of a composite implant was evaluated. Both sides of the ePTFE used in Bard COMPOSIX® products (COMPOSIX E/X and COMPOSIX Kugel) were coated and attached to uncoated polypropylene mesh using PTFE monofilament. This configuration allows for antimicrobial activity on both sides of the product (due to release of silver ions), but the ingrowth part of the composite, the polypropylene mesh, was not coated directly to alleviate negative effects on tissue ingrowth.

One finding of the histological analysis of the various coated DULEX implants after 14 days was that the silver coating level originally determined by the zone of inhibition testing might have been higher than necessary. At 14 days, a dark layer of the silver coating was visible on the surface of the mesh and particulates containing silver were found in the cells adjacent to the surface. This suggested that there was possibly an excess of silver coating, as the goal was to provide antimicrobial protection for 10 days following implantation. In patches with a lower initial silver level in the coating, the coating was not visible and the particulates in the cells were not readily seen. Because of this, alternative in vitro test methods of the silver coatings were investigated. The original in vitro tests involved repeated removal of the coated materials from agar plates on which they had formed zones to new plates with fresh bacterial lawns. In some cases, silver was visibly left behind on the plate from which it was removed, reducing its duration of activity on subsequent plates. Although the coating could be rubbed away from the surface by contact with tissues, the silver would still be present in the area of the prosthesis, and able to still have an antimicrobial effect. It should be noted that the DUALMESH Plus material, which carries a claim of 10 days of activity, only reached 6 days of activity with the plate-to plate method.

An alternative test was developed to expose the coated material to an agar plate at body temperature for a specified time period. The coating would be able to diffuse into the agar over time, and would then, at the end of that time period, be challenged with organisms in the zone of inhibition test. The test used Mueller-Hinton agar (MHA), a standard agar often used to test antimicrobial susceptibility of organisms. In these tests, the silver-coated ePTFE material that serves as the barrier surface of the composite meshes was tested. The results of the MHA testing on silver-coated ePTFE samples are summarized in Table 3.

TABLE 3
Results of Mueller-Hinton Agar Test for Duration of Activity

| | Available | | Available | Antimicrobial Activity | |
|---|---|---|---|---|---|
| | Silver | Total Silver | Silver | Duration | Zone Size |
| Material | (mg/cm²) | (mg/cm²) | (%) | (days) | (mm) |
| ePTFE | 0.09 | 0.245 | 36.7 | 10 | 8.7 |
| ePTFE | 0.06 | 0.225 | 26.7 | 10 | 5.3 |
| ePTFE | 0.057 | 0.149 | 38.3 | 10 | 5.7 |
| ePTFE* | 0.046 | 0.096 | 47.9 | 10 | 6.5 |
| ePTFE | 0.04 | 0.235 | 17.0 | 10 | 0.3 |
| ePTFE* | 0.030 | 0.093 | 32.3 | 10 | 7.6 |
| ePTFE | 0.032 | 0.113 | 28.2 | 10 | 1.3 |
| ePTFE* | 0.023 | 0.091 | 25.3 | 10 | 1.8 |
| ePTFE | 0.016 | 0.086 | 18.8 | 7 | 1.5 |
| ePTFE* | 0.011 | 0.073 | 15.1 | 10 | 2.3 |
| ePTFE | 0.01 | 0.061 | 16.3 | 7 | 1.3 |
| DualMesh | NA | NA | NA | 10 | 0.7 |
| Plus | | | | | |

*these levels correspond to the study in Table 4.

A number of the coating levels tested provided the 10-day duration of activity desired using the new test method. It was then necessary to determine which, if any, of these coating levels would not have a negative impact on tissue ingrowth. Sheets of ePTFE were coated on both sides with various levels of silver and then assembled with polypropylene mesh and PTFE stitching to form composite test articles. The polypropylene mesh was not coated. The test articles were sterilized and tested in vivo. The first test included three silver levels and two controls: Bard COMPOSIX and Gore DUALMESH Plus. The materials were implanted in rabbits and removed at 7 and 14 days post-implantation for histological analysis. A second study was later done to test three additional silver levels. The results of the histological analyses for both studies are summarized in Table 4. The results presented Table 4 describe the inflammatory response and collagen formation on the mesh side of the medical implant/article, where ingrowth is intended to occur.

TABLE 4
Results of Histological Analyses of Silver-Coated
COMPOSIX E/X Mesh, Uncoated Control COMPOSIX Mesh,
and DUALMESH Plus Implanted at Various Time Periods

| | Available | Total | | Implant | | |
|---|---|---|---|---|---|---|
| | Silver | Silver | Available | Duration | Inflammatory | |
| Material | (mg/cm²) | (mg/cm²) | Silver (%) | (days) | Response | Collagen Formation |
| Composix | 0.007 | 0.011 | 63.6% | 7 | Occasional giant cells | Fibrosis abundant in outer half of the |
| E/X | | | | | on the mesh side of | mesh (away from ePTFE), new |
| | | | | | the ePTFE layer. | collagen formation evident in inner |
| | | | | | | half of mesh. |
| Composix | 0.007 | 0.011 | 63.6% | 14 | Occasional giant cells | Mesh space filled with fibrous tissue. |
| E/X | | | | | on the mesh side of | |
| | | | | | the ePTFE layer. | |
| Composix | 0.011 | 0.073 | 15.1% | 7 | Macrophages and | No apparent difference from |
| E/X* | | | | | giant cells. | uncoated control. |
| Composix | 0.024 | 0.033 | 72.7% | 7 | Giant cells on mesh | Meshwork almost acellular. Fibrosis |
| E/X | | | | | side of ePTFE. | limited to outer portion of mesh |
| | | | | | | (away from ePTFE). |
| Composix | 0.024 | 0.033 | 72.7% | 14 | Giant cells line the | Well-developed fibrosis within the |
| E/X | | | | | surface of the ePTFE | mesh. |
| | | | | | on the mesh side. | |
| Composix | 0.023 | 0.091 | 25.3% | 7 | Macrophages and | No apparent difference from |
| E/X* | | | | | giant cells. | uncoated control. |
| Composix | 0.030 | 0.093 | 32.3% | 7 | Macrophages and | No apparent difference from |
| E/X* | | | | | giant cells. | uncoated control. |
| Composix | 0.039 | 0.073 | 53.4% | 7 | Frequent giant cells | Mesh volume filled with extracellular |
| E/X | | | | | on mesh side of | matrix sparsely populated with |
| | | | | | ePTFE. | spindle-shaped cells. |
| Composix | 0.039 | 0.073 | 53.4% | 14 | Occasional giant cells | New collagen formation noted, |
| E/X | | | | | on the ePTFE surface | ranging from abundant in some areas |
| | | | | | on the mesh side. | to moderate in others. |
| Composix | 0.046 | 0.096 | 47.9% | 7 | Macrophages and | No apparent difference from |
| E/X* | | | | | giant cells. | uncoated control. |
| Composix | 0.112 | 0.311 | 36.0% | 7 | Giant cells and | Robust fibrotic response in the outer |
| E/X | | | | | macrophages on | half (away from ePTFE) of the mesh. |
| | | | | | mesh side of ePTFE. | Sparse cellularity within inner half |
| | | | | | | (near ePTFE) of mesh. |
| Composix | 0.112 | 0.311 | 36.0% | 14 | Macrophages and | Fibrotic response in the outer half of |
| E/X | | | | | giant cells, some | the mesh. |
| | | | | | appear to be | |
| | | | | | degenerating | |
| Composix | 0.169 | 0.442 | 38.2% | 7 | Macrophages, some | Robust fibrotic response in the outer |
| E/X | | | | | appear to be | half (away from ePTFE) of the mesh. |
| | | | | | degenerating | Sparse cellularity within inner half |
| | | | | | | (near ePTFE) of mesh. |
| Composix | 0.169 | 0.442 | 38.2% | 14 | Thickened tissue | Robust fibrotic response surrounding |
| E/X | | | | | response, comprised | the mesh monofilaments in the outer |
| | | | | | mostly of | half of the mesh. Sparse matrix with |
| | | | | | macrophages and | occasional giant cells in the inner half |
| | | | | | giant cells. Evidence | of the mesh. |
| | | | | | of cell lysis. | |
| Composix | 0.189 | 0.513 | 36.8% | 7 | Sparse cellular | Sparse extracellular matrix |
| E/X | | | | | response; giant cells | throughout mesh; discontinuous fibrin |
| | | | | | | network. |
| Composix | 0.189 | 0.513 | 36.8% | 14 | Macrophages and | Modest fibrotic response arising from |
| E/X | | | | | frequent giant cells | the underlying peritoneal surface. |
| Composix | N/A | N/A | N/A | 7 | Occasional giant cells | Mesh filled with sparsely populated |
| Control | | | | | and macrophages at | fibrin network. |
| | | | | | ePTFE surface on | |
| | | | | | mesh side. | |
| Composix | N/A | N/A | N/A | 14 | Occasional giant cells | More than half of mesh filled with |
| Control | | | | | and macrophages at | dense fibrous tissue, the rest filled |
| | | | | | ePTFE surface on | with a loose, sparsely populated |
| | | | | | mesh side. | extracellular matrix. |
| DualMesh | N/A | N/A | N/A | 7 | Macrophages within | Sparsely cellular with fibrin network |
| Plus | | | | | fibrin meshwork on | between the mesh. |
| | | | | | ingrowth side. | |
| DualMesh | N/A | N/A | N/A | 14 | Chronic inflammatory | Fibrous tissue formation. |
| Plus | | | | | response with | |
| | | | | | macrophages and | |
| | | | | | giant cells. | |

The COMPOSIX controls were marked by signs of an inflammatory response at 7 days, with occasional giant cells and macrophages noted. The mesh was filled with a sparsely populated fibrin network. By 14 days, the mesh was more than half filled with dense fibrous tissue, with the rest containing a sparsely populated extracellular matrix, and the inflammatory response about the same as it was at 7 days. For three of the six coating levels implanted in these two studies, the tissue response was basically the same as that of the uncoated control mesh. However, signs of a negative effect of the coating—cellular degeneration and lysis—marked the three highest levels.

A third implant study was begun to investigate the differences in the host response to four additional silver coating levels. COMPOSIX E/X-type samples coated with silver were implanted in the rat abdomen and then excised for histological analysis 7 days later. There were no apparent differences in the healing response within the monofilament meshwork between the coated ePTFE surface and the abdominal wall for any of the test articles when compared with the control composite patch. It is notable, however, that the tissue response adjacent to the coated ePTFE surfaces on the visceral side did differ from the control article. The tissue response on the visceral side was thicker with more macrophages than that of the control article (fibrotic with more spindle-shaped cells than macrophages).

### DISCUSSION

The impact of the silver coating on tissue ingrowth into Bard DULEX Mesh appears to be significant, even at low levels. At the lowest level tested, the formation of new collagen within the ingrowth side fibers was low relative to an uncoated control at 14 days. At 7 days, a higher level tested (0.0227/0.040 mg/cm² available/total silver), the collagen formation was closer to that of the control. However, macrophages with abnormal morphology were noted.

With the composite test implants coated with silver on the ePTFE barrier layer only, it appears that a range of silver levels (up to 0.046/0.096 mg/cm² available/total silver in these studies) can be applied without a negative effect on the tissue response and new collagen formation. In the studies described in Table 4, seven of the silver levels resulted in tissue responses and collagen formation essentially equivalent to the uncoated control. In that study, it was also apparent that the effect of the silver coating on the tissue response is greater immediately adjacent to the coated surface. Therefore it seems that in the composite meshes, however, where the tissue ingrowth is intended to occur at a relatively large (compared with the visceral side) distance from the coated surface, the impact of the silver coating was minimal compared to the coated DULEX mesh, where the coating is applied directly to the ingrowth surface.

The studies described here show that not only is it possible to coat the ePTFE barrier surface of the composite with various levels of silver without inhibiting tissue integration and ingrowth into the polypropylene mesh, it is possible to do so with levels of silver that provide the desired antimicrobial effect.

By coating the ePTFE barrier component of the composite meshes while leaving the mesh ingrowth component uncoated, it is possible to achieve the antimicrobial effect required for a soft tissue implant while preserving the normal implant performance. By comparing the performance of the coated composite meshes with the effect of the silver coating on the tissue response at the visceral surface of the composite meshes and the ingrowth side of the DULEX mesh, it appears that by not coating the ingrowth surface directly, the mesh performance can be enhanced by the antimicrobial substance without sacrificing tissue ingrowth.

Other aspects of the invention will be clear to the skilled artisan and need not be repeated here. Each reference cited herein is incorporated by reference in its entirety.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

1. A prosthetic device for repairing, reconstructing and/or augmenting a defect or a weakness in a wall of a muscle, tissue or organ, comprising:
a first portion that is tissue infiltratable;
a second portion that is a barrier to tissue ingrowth; and
an effective amount of at least one antimicrobial substance incorporated with said second portion;
wherein the effective amount of said at least one substance is not incorporated with said first portion.

2. The prosthetic device of claim 1 wherein the antimicrobial substance is silver or a silver salt.

3. The prosthetic device of claim 1 wherein said at least one antimicrobial substance provides a first level of impairment of tissue ingrowth, and further comprising a second antimicrobial substance that provides a second level of impairment of tissue ingrowth which is no more than said first level, said second antimicrobial substance being incorporated with said first portion.

4. The prosthetic device of claim 1 wherein the second antimicrobial substance does not substantially impair tissue ingrowth.

5. The prosthetic device of claim 1 wherein said first portion has a sheet-like configuration.

6. The prosthetic device of claim 1 wherein said second portion has a sheet-like configuration.

7. The prosthetic device of claim 1 wherein said first portion has a sheet-like configuration and said second portion has a sheet-like configuration.

8. The prosthetic device of claim 1 or claim 7 wherein said first portion and said second portion are permanently attached together.

9. The prosthetic device of claim 1 wherein said first portion is a knitted sheet.

10. The prosthetic device of claim 1 wherein said second portion is a sheet of ePTFE.

11. The prosthetic device of claim 6 or claim 7 wherein said sheet-like second portion has a top surface and a bottom surface, and said at least one antimicrobial substance is substantially incorporated with at least one of said top and bottom surfaces.

12. The prosthetic device of claim 11 wherein said at least one antimicrobial substance is incorporated with both of said top and bottom surfaces.

13. The prosthetic device of claim 1 wherein said first portion and said second portion are formed of the same material.

14. The prosthetic device of claim 13 wherein said same material is ePTFE.

15. A prosthetic device for repairing, reconstructing and/or augmenting a defect or a weakness in a wall of a muscle, tissue or organ, comprising:
a flexible, biocompatible and implantable porous fabric sheet having a first surface;
a flexible, biocompatible and implantable substantially non-porous barrier sheet having a second surface;
said porous fabric sheet and said barrier sheet being permanently attached together with said first surface facing said second surface;
an effective amount of at least one antimicrobial substance being incorporated with said substantially non-porous barrier sheet, said porous fabric sheet not incorporating an effective amount of said at least one antimicrobial substance.

16. The prosthetic device of claim 15 wherein the antimicrobial substance is silver or a silver salt.

17. The prosthetic device of claim 15 wherein said at least one antimicrobial substance provides a first level of impairment of tissue ingrowth, and further comprising a second antimicrobial substance that provides a second level of impairment of tissue ingrowth which is no more than said first level, said second antimicrobial substance being incorporated with said porous fabric sheet.

18. The prosthetic device of claim 17 wherein the second antimicrobial substance does not substantially impair tissue ingrowth.

19. The prosthetic device of claim 15 wherein said porous fabric sheet is a knitted sheet.

20. The prosthetic device of claim 15 wherein said substantially non-porous barrier sheet is a sheet of ePTFE.

21. The prosthetic device of claim 15 wherein said substantially non-porous barrier sheet has a top surface and a bottom surface, and said at least one antimicrobial substance is substantially incorporated with at least one of said top and bottom surfaces.

22. The prosthetic device of claim 21 wherein said at least one antimicrobial substance is incorporated with both of said top and bottom surfaces.

23. The prosthetic device of claim 15 wherein said porous fabric sheet and said substantially non-porous barrier sheet are formed of the same material.

24. The prosthetic device of claim 23 wherein said same material is ePTFE.

25. A prosthetic device for repairing, reconstructing and/or augmenting a defect or a weakness in a wall of a muscle, tissue or organ, comprising:
a first portion that is tissue infiltratable;
a second portion that is a barrier to tissue ingrowth;
an effective amount of at least one antimicrobial substance incorporated in said prosthetic device that does not detrimentally impair tissue ingrowth into and/or around said first portion.

26. The prosthetic device of claim 25 wherein the antimicrobial substance is silver or a silver salt.

27. The prosthetic device of claim 25 wherein said first portion has a sheet-like configuration.

28. The prosthetic device of claim 25 wherein said second portion has a sheet-like configuration.

29. The prosthetic device of claim 25 wherein said first portion has a sheet-like configuration and said second portion has a sheet-like configuration.

30. The prosthetic device of claim 25 or claim 29 wherein said first portion and said second portion are permanently attached together.

31. The prosthetic device of claim 25 wherein said first portion is a knitted sheet.

32. The prosthetic device of claim 25 wherein said second portion is a sheet of ePTFE.

33. The prosthetic device of claim 28 or claim 29 wherein said sheet-like second portion has a top surface and a bottom surface, and said at least one antimicrobial substance is substantially incorporated with at least one of said top and bottom surfaces.

34. The prosthetic device of claim 33 wherein said at least one antimicrobial substance is incorporated with both of said top and bottom surfaces.

35. The prosthetic device of claim 25 wherein said first portion and said second portion are formed of the same material.

36. The prosthetic device of claim 35 wherein said same material is ePTFE.

37. A method for repairing, reconstructing and/or augmenting a defect or a weakness in a wall of a muscle, tissue or organ comprising
implanting into a subject in need of such treatment a prosthetic device of any of claims 1-36 to repair, reconstruct and/or augment the defect or weakness in the wall of the muscle, tissue or organ.
